(19) United States
(12) Patent Application Publication
(10) Pub. No.: US 2023/0104998 A1
(43) Pub. Date: Apr. 6, 2023

(54) SINGLE-MOLECULE PROTEIN AND PEPTIDE SEQUENCING

(71) Applicant: Massachusetts Institute of Technology, Cambridge, MA (US)

(72) Inventors: Daniel Masao Estandian, Cambridge, MA (US); Edward Stuart Boyden, Chestnut Hill, MA (US); Jacob Joshua Lee Rodriguez, Somerville, MA (US)

(73) Assignee: Massachusetts Institute of Technology, Cambridge, MA (US)

(21) Appl. No.: 17/949,788

(22) Filed: Sep. 21, 2022

Related U.S. Application Data
(60) Provisional application No. 63/247,011, filed on Sep. 22, 2021.

(51) Int. Cl. G01N 33/68; G01N 33/58

(52) U.S. Cl. CPC G01N 33/6818; G01N 33/58; G01N 2458/00

(57) ABSTRACT

The present description provides methods, assays and reagents for linearly expanding a peptide. The methods and/or linear expanded peptide described herein have several uses such as, but not limited to, peptide (protein) sequencing, high-resolution interrogation of the proteome and enabling ultrasensitive diagnostics critical for early detection of diseases.

### RELATED APPLICATIONS

This application claims the benefit under 35 U.S.C. §119(e) of U.S. Provisional application serial number 63/247,011 filed Sep. 22, 2021, the disclosure of which is incorporated by reference herein in its entirety.

### GOVERNMENT SUPPORT

This invention was made with Government support under Grant No. HG008525 awarded by the National Institutes of Health (NIH). The Government has certain rights in the invention.

### BACKGROUND

Proteins serve critical structural and dynamic functional roles at the cellular level of all living organisms. Understanding protein contribution to biological function is critical and rests on having appropriate technologies for quantification and identification. The central dogma of molecular biology, information flow from DNA to RNA to protein, has been studied for decades because these molecules are critical to cell function and diversity. The advent of polymerase chain reaction (PCR) amplification of nucleic acid was pivotal in advancing the high-throughput molecular interrogation and analysis of DNA and RNA at the whole-genome and transcriptome level. In contrast, studying proteins has lagged technologically since there is no equivalent of PCR to amplify and detect low-copy number proteins. Instead, protein sequencing and identification methods have relied on ensemble measurements from many cells, which masks cell-to-cell variations. Although some researchers have turned to transcriptomics as a proxy to the protein composition within cells, it is critical to note that gene expression at the transcriptomic level weakly correlates with the proteomic profile due to variability in translational efficiency of different mRNAs, and the difference between mRNA and protein lifetimes. In addition, post-translational modifications also result in significant variability of protein abundance and their primary sequence with respect to the transcriptome. Vital biological processes such as synaptic plasticity, metabolic signaling pathways and stem cell differentiation, all depend on protein expression. Many diseases also originate from genetic mutations that are in turn translated to a single aberrant protein or a set of aberrant proteins. Diseases such as cancer and neurodegeneration tend to have triggered mutations of unclear origins and polygenic interactions. They can be best understood and addressed at the proteomic level, since their pathology is directly related to disrupted proteostasis at the cellular level.

Advancements in proteomics have lagged behind while DNA sequencing has rapidly advanced the study of genomics primarily due to technologies that allow for high-throughput sequencing. Current methodologies for studying proteins include Mass Spectrometry, Edman sequencing and Immunohistochemistry (IHC).

Mass spectrometry enables protein identification and quantification based on the mass/charge ratio of peptide fragments, which can be bioinformatically mapped back to a genomic database. Although this technique has made significant advancements, it has yet to quantify a complete set of proteins from a biological system. The technology exhibits attomole detection sensitivities for whole proteins and subattomole sensitivities after fractionation. The sensitivity of mass spectrometry is limiting since low copy-number proteins that make up about 10% of mammalian protein expression remain undetected and are functionally important despite low abundance.

The other method used for protein sequencing is the Edman degradation reaction. Edman degradation allows for sequential and selective removal of single N-terminal amino acids, subsequently identified via HPLC, High-Performance Liquid Chromatography. Edman protein sequencing is a proven method to selectively remove the first N-terminal amino acid for identification in which phenyl isothiocyanate (PITC) is used to conjugate to the N-terminal amino acid, then upon acid and heat treatment, the PITC-labeled N-terminal amino acid is removed. Although Edman sequencing can have 98% efficiency, a major drawback is that it is inherently low throughput, requiring a single highly purified protein and not applicable to systems-wide biology. Both Edman degradation and mass spectrometry can sequence proteins but lack single molecule sensitivity and do not provide spatial information of proteins in the context of cells.

In regard to spatial information, immunohistochemistry is a protein identification method that allows us to visualize cellular localization of proteins but does not provide sequence information. Immunohistochemistry involves the identification of proteins via recognition with fluorophore-conjugated antibodies. This approach excludes protein sequence information but can identify proteins and their respective localizations. A major limitation is the scalability, since even the perfect construction of specific antibodies for every protein in the proteome would require around 25,000 antibodies and, ~6250 rounds of four-color imaging. Any 1-to-1 protein-tagging scheme will likely fail to scale to the entire proteome.

A major obstacle in protein sequencing is the lack of natural enzymes and biomolecules that probe amino acids on a peptide. For example, protein amplification processes analogous to PCR for nucleic acids do not exist, so the approach to sequencing via single-molecule strategies is appropriate, requiring the detection of individual amino acids.

Current proposed approaches to single molecule protein sequencing rely either on fluorescent read-out via covalent chemical modifications of peptide or protein residues, probing with N-terminal-specific amino-acid binders (NAABs), or translocating peptides through a nanopore with a voltage applied across the membrane. Chemical modifications of amino acids on the internal peptide chain may be vulnerable to low efficiencies due to steric hindrance caused by adjacent chemical labels, and there are also a limited number of available reactive amino acids and chemistries for labeling of all 20 amino acids. A major issue using nanopores for protein sequencing can be attributed to the non-uniform charge distribution of amino acid residues and the analytical challenge of deconvolving electric recordings to discriminate between amino acids.

The lack of technology for high-resolution protein-level analyses represents a significant gap in advancing important biological research.

### SUMMARY OF THE INVENTION

The invention provides a method for linearly expanding a peptide. As used herein, linear expanding a peptide means that the distance between amino acids of a peptide is increased (expanded) while maintaining the sequence of the peptide. The terms “expanded peptide” or “a linearly expanded peptide” are used interchangeably herein to refer to any peptide produced by any of the methods described herein.

In embodiments, the method comprises contacting the peptide with a binding element (also referred to herein as “the element”) that interacts with a terminal amino acid or a terminal amino acid derivative of the peptide to form an element-peptide complex, tethering the element-peptide complex to a substrate; cleaving the element-peptide complex from the peptide thereby providing an element-amino acid complex bound to the substrate. In embodiments, the element comprises a linker wherein the linker provides an attachment point for the next amino acid of the peptide. In embodiments, the method comprises attaching a linker to the element of the element-amino acid complex wherein the linker provides an attachment point for the next amino acid of the peptide. The “next amino acid of the peptide” is now the terminal amino acid and can be contacted with an element to form element-amino acid complex. Two or more element-amino acid complexes can be connected through the linker. In one embodiment, the peptide is affixed to a substrate.

In embodiments, the method is repeated one or more times. For example, after the terminal amino acid of the peptide has been removed, the peptide is again contacted with the element to form a further element-peptide complex with the next, now terminal amino acid, of the peptide; tethering the further element-peptide complex to the linker of the previous element; and cleaving the further element-peptide complex from the peptide. In embodiments, the element comprises a linker wherein the linker provides an attachment point for the next amino acid of the peptide. In embodiments, a further linker is attached to the further element-amino acid complex. The linker provides an attachment point for the use of the method on the next amino acid of the peptide. The “next amino acid of the peptide” is now the terminal amino acid and can be contacted with an element to form element-amino acid complex. Two or more element-amino acid complexes can be connected through the linker. In some embodiments, the method is repeated until a portion of the peptide is expanded. In embodiments, the method is repeated until the entire peptide is expanded. In some embodiments, the method also includes contacting one or more additional peptides (such that two or more peptides are contacted) with a binding element that interacts with a terminal amino acid or a terminal amino acid derivative of the peptides to form element-peptide complexes; tethering the element-peptide complexes to a substrate; and cleaving the element-peptide complexes from the peptide resulting in element-amino acid complexes bound to the substrate; thereby linearly expanding the two or more peptides. In some embodiments, before the contacting step the two or more peptides are independently affixed to the substrate. In some embodiments, the two or more peptides are different from each other.

The invention also provides a method for linearly expanding two or more peptides. For example, the distance between amino acids of two or more peptides in a sample can be expanded (increased) while maintaining the sequences (i.e., order of amino acids) of the two or more peptides. In embodiments, the method comprises independently affixing the two or more peptides to a substrate; contacting the peptides with a binding element that interacts with the terminal amino acid or terminal amino acid derivative of each peptide to form element-peptide complexes, tethering the element-peptide complexes to the substrate; cleaving the element-peptide complexes from the peptides thereby providing element-amino acid complexes bound to the substrate. In some embodiments, the element comprises a linker wherein the linker provides an attachment point for the next amino acid of the peptide. In embodiments, the method comprises attaching a linker to the element of the element-amino acid complexes wherein the linker provides an attachment point for the next amino acid of the peptide. The “next amino acid of the peptide” is now the terminal amino acid and can be contacted with an element to form element-amino acid complex. Two or more element-amino acid complexes can be connected through the linker.

The invention also provides a method for linearly expanding of at least a portion of a peptide. In embodiments, the method comprises contacting the peptide with a binding element that interacts with a terminal amino acid or terminal amino acid derivative of the peptide to form an element-peptide complex, tethering the element-peptide complex to a substrate; cleaving the element-peptide complex from the peptide to form an element-amino acid complex bound to the substrate, wherein the element comprises a linker that provides an attachment point for the next amino acid of the peptide or such a linker is added to the element of the element-amino acid complex; again contacting the peptide with a binding element to form a further element-peptide complex with the next, now terminal amino acid of the peptide, tethering the further element-peptide complex to the linker of the previous element-amino acid complex; and cleaving the element-peptide complex from the peptide thereby providing linked element-amino acid complexes bound to the substrate; wherein the distance between the amino acids has been increased. In embodiments, the element of the further element-amino acid complex comprises a linker wherein the linker provides an attachment point for the next amino acid of the peptide. In embodiments, the method comprises attaching a linker to the element of the further element-amino acid complex wherein the linker provides an attachment point for the next amino acid of the peptide. The “next amino acid of the peptide” is now the terminal amino acid and can be contacted with an element to form element-amino acid complex. Two or more element-amino acid complexes can be connected through the linker. In embodiments, the method is repeated one or more times. In embodiments, the method comprises the linearly expanding all amino acids of the peptide. In some embodiments, the method also includes linearly expanding of at least a portion of a one or more additional peptides (also referred to herein as expanding of at least a portion of two or more peptides) comprising contacting the one or more additional peptides with a binding element that interacts with a terminal amino acid or terminal amino acid derivative of the peptides to form element-peptide complexes; tethering the element-peptide complexes to a substrate; cleaving the element-peptide complexes from the peptides to form element-amino acid complexes bound to the substrate, wherein the element comprises a linker that provides an attachment point for a next amino acid of the peptides or such a linker is added to the element of the element-amino acid complexes; contacting the peptides with a binding element to form a further element-peptide complexes with the next, now terminal amino acid of the peptide, tethering the further element-peptide complexes to the linker of the element-amino acid complexes; and cleaving the element-peptide complexes from the peptides thereby providing linked element-amino acid complexes bound to the substrate; wherein the distance between the amino acids in the peptides has been increased, thereby linearly expanding at least a portion of the two or more peptides. In some embodiments, the method also includes performing steps of the aforementioned method on one or more additional peptides, thereby linearly expanding at least a portion of the two or more peptides.

The invention also provides a method for linearly expanding at least a portion of two or more peptides in a sample independently affixed attachment points on a substrate. In embodiments, the method comprises contacting the two or more peptides with a binding element that interacts with a terminal amino acid or terminal amino acid derivative of each peptide to form element-peptide complexes, tethering the element-peptide complexes to the substrate; cleaving the element-peptide complexes from the peptides to form element-amino acid complexes bound to the substrate, wherein the element comprises a linker that provides an attachment point for the next amino acid of the peptide or such a linker is added to the element of the element-amino acid complex; again contacting the peptides with a binding element to form a further element-peptide complex with the next, now terminal amino acid of the peptide, tethering the further element-peptide complex to the linker of the previous element-amino acid complex bound to the substrate; and cleaving the element-peptide complexes from the peptides thereby providing linked element-amino acid complexes bound to the substrate; wherein the distance between the amino acids has been increased. The “next amino acid of the peptide” is now the terminal amino acid and can be contacted with an element to form element-amino acid complex. Two or more element-amino acid complexes can be connected through the linker.

In embodiments, the elements of the further element-amino acid complexes comprise a linker wherein the linker provides an attachment point for the next amino acid of the peptides. In embodiments, the method comprises attaching a linker to the elements of the further element-amino acid complexes wherein the linker provides an attachment point for the next amino acid of the peptide. The “next amino acid of the peptide” is now the terminal amino acid and can be contacted with an element to form element-amino acid complex. Two or more element-amino acid complexes can be connected through the linker. In embodiments, the method is repeated one or more times. In embodiments, the method comprises the linearly expanding all amino acids of the peptide.

Once a portion of the peptide, or all amino acids of the peptide, has been expanded by any of the processes described herein, the expanded peptide can be sequenced by any suitable method known in the art. Detection methods for protein sequencing include, but are not limited to, nanopores, ionic current nanopores, tunneling current nanopores, atomic force microscopy, protein binder, aptamer binder, multimeric binder, DNA-paint, and chemical conjugations.

The invention also provides an element-amino acid complex. In embodiments, the element-amino acid complex comprises a binding element bound to one of 20 natural proteinogenic amino acids; a binding element bound to a post-translationally modified amino acid; or a binding element bound to a derivative of an amino acid of a peptide.

The invention also provides an element-amino acid complex binder. In embodiments, the element-amino acid complex binder comprises a binder that binds to one or a subgroup of the 20 natural proteinogenic amino acids complexed with the element; a binder that binds to a one or a subgroup of post-translationally modified amino acids complexed with the element; or a binder that binds to a derivative of an amino acid of a peptide.

In some embodiments, the element-amino acid complex binder comprises a binder that binds to one of 20 natural proteinogenic amino acids complexed with the element; a binder that binds to a post-translationally modified amino acids complexed with the element; or a binder that binds to a derivative of an amino acid of a peptide.

In certain embodiments of any of the aforementioned compounds, compositions, and/or methods described herein, the binding element is a ClickT compound as described herein.

According to one aspect of the invention, a method for linearly expanding a peptide is provided, the method including: contacting the peptide with a binding element that interacts with a terminal amino acid or a terminal amino acid derivative of the peptide to form an element-peptide complex; tethering the element-peptide complex to a substrate; and cleaving the element-peptide complex from the peptide resulting in an element-amino acid complex bound to the substrate. In some embodiments, the method also includes performing the method on one or more additional peptides thereby linearly expanding two or more peptides. In certain embodiments, the two or more peptides are different from each other. According to another aspect of the invention, a method for linearly expanding two or more peptides is provided, the method including: contacting the two or more peptides with a binding element that interacts with the terminal amino acid or terminal amino acid derivative of the two or more peptides to form element-peptide complexes, tethering the element-peptide complexes to the substrate; and cleaving the element-peptide complexes from the peptides resulting element-amino acid complexes bound to the substrate. In some embodiments of any aforementioned aspect of the invention, the binding element comprises a linker that provides an attachment point for the next amino acid of the peptide. In some embodiments of a method of any aforementioned aspect of the invention, the next amino acid is the terminal amino acid of the peptide after the peptide has been cleaved from the element-peptide complex. In certain embodiments, a method of any aforementioned aspect of the invention also includes attaching to the binding element linker the next amino acid of the peptide after the peptide is cleaved from the element-peptide complex, resulting in the next amino acid of the peptide being part of an element-amino acid complex. In certain embodiments of a method of any aforementioned aspect of the invention, the binding element comprises a linker. In some embodiments of a method of any aforementioned aspect of the invention, the method also includes attaching a linker to the element of further element-amino acid complexes wherein the linker provides an attachment point for the next amino acid of the peptide. In some embodiments of a method of any aforementioned aspect of the invention, the next amino acid of the peptide is a terminal amino acid of the peptide following the cleaving of the peptide from the element-peptide complex. In some embodiments of a method of any aforementioned aspect of the invention, the next amino acid of the peptide is part of element-amino acid complex. In certain embodiments of a method of any aforementioned aspect of the invention, the method also includes to the linker the next amino acid of the peptide that has been cleaved from the element-peptide complex, resulting in the next amino acid of the peptide being part of element-amino acid complex. In certain embodiments of a method of any aforementioned aspect of the invention, the binding element binds to an N-terminal amino acid or N-terminal amino acid derivative of the peptide to form an element-peptide complex. In certain embodiments of a method of any aforementioned aspect of the invention, the binding element binds to a C-terminal amino acid or C-terminal amino acid derivative of the peptide to form an element-peptide complex. In some embodiments of a method of any aforementioned aspect of the invention, prior to tethering and/or cleaving excess and/or unbound binding element is washed away. In some embodiments of a method of any aforementioned aspect of the invention, the method is repeated one or more times. In certain embodiments of a method of any aforementioned aspect of the invention, the method is repeated for all amino acids of the peptide. In certain embodiments of a method of any aforementioned aspect of the invention, the steps of the method are repeated one or more times. In some embodiments of a method of any aforementioned aspect of the invention, the steps of contacting, tethering, cleaving, and the attaching a linker to the element of the further element-amino acid complexes wherein the linker provides an attachment point for the next amino acid of the peptide are repeated for all amino acids of the peptide. In some embodiments of a method of any aforementioned aspect of the invention, prior to the step of contacting, the peptide is affixed to a substrate. In certain embodiments of a method of any aforementioned aspect of the invention, prior to the step of contacting, the two or more peptides are independently affixed to a substrate. In certain embodiments of a method of any aforementioned aspect of the invention, the two or more peptides are the same as each other. In some embodiments of a method of any aforementioned aspect of the invention, at least two of the two or more peptides are different from each other. In some embodiments of a method of any aforementioned aspect of the invention, all of the two or more peptides are different from each other. In certain embodiments of a method of any aforementioned aspect of the invention, the peptide is affixed to the substrate through the C′-terminal carboxyl group or a side chain functional group of the peptide. In some embodiments of a method of any aforementioned aspect of the invention, the peptide is affixed to the substrate through the N′-terminal carboxyl group or a side chain functional group of the peptide. In some embodiments of a method of any aforementioned aspect of the invention, the peptide is covalently affixed to the substrate. In certain embodiments of a method of any aforementioned aspect of the invention, the substrate is optically transparent. In certain embodiments of a method of any aforementioned aspect of the invention, the substrate comprises a functionalized surface. In some embodiments of a method of any aforementioned aspect of the invention, the functionalized surface is selected from the group consisting of an azide functionalized surface, a thiol functionalized surface, alkyne, DBCO, maleimide, succinimide, tetrazine, TCO, vinyl, methylcyclopropene, a primary amine surface, a carboxylic surface, a DBCO surface, an alkyne surface, and an aldehyde surface. In some embodiments of a method of any aforementioned aspect of the invention, the method also includes the steps of contacting, tethering, cleaving, and the attaching a linker are repeated on one or more additional peptides thereby linearly expanding the two or more peptides. In some embodiments of a method of any aforementioned aspect of the invention, the method also includes sequencing the linearly expanded peptide. In certain embodiments of a method of any aforementioned aspect of the invention, the method also includes the sequence of the peptide to a reference-protein-sequence database. In certain embodiments of a method of any aforementioned aspect of the invention, the method also includes comparing the sequences of each peptide, grouping similar peptide sequences and counting the number of instances of each similar peptide sequence. In some embodiments of a method of any aforementioned aspect of the invention, the peptide or the two or more peptides are from a sample. In some embodiments of a method of any aforementioned aspect of the invention, the sample includes a biological fluid, cell extract, tissue extract, or a mixture of synthetically synthesized peptides. In some embodiments of a method of any aforementioned aspect of the invention, the sample is a mammalian sample. In certain embodiments of a method of any aforementioned aspect of the invention, the sample is a human sample. In certain embodiments of a method of any aforementioned aspect of the invention, the binding element is a ClickT compound.

According to another aspect of the invention, a method for linearly expanding of at least a portion of a peptide is provided, the method including: contacting the peptide with a binding element that interacts with a terminal amino acid or terminal amino acid derivative of the peptide to form an element-peptide complex; tethering the element-peptide complex to a substrate; leaving the element-peptide complex from the peptide to form an element-amino acid complex bound to the substrate, wherein the element comprises a linker that provides an attachment point for the next amino acid of the peptide or such a linker is added to the element of the element-amino acid complex; contacting the peptide with a binding element to form a further element-peptide complex with the next, now terminal amino acid of the peptide, tethering the further element-peptide complex to the linker of the element-amino acid complex; and cleaving the element-peptide complex from the peptide thereby providing linked element-amino acid complexes bound to the substrate; thereby increasing the distance between the amino acids of the peptide. In some embodiments, the method also includes performing the steps of the aforementioned method on one or more additional peptides, thereby linearly expanding at least a portion of the two or more peptides. According to another aspect of the invention, a method for linearly expanding at least a portion of two or more peptides is provided, the method including contacting the two or more peptides with a binding element that interacts with a terminal amino acid or terminal amino acid derivative of the peptides to form element-peptide complexes, tethering the element-peptide complexes to the substrate; cleaving the element-peptide complexes from the peptides to form element-amino acid complexes bound to the substrate, wherein the element comprises a linker that provides an attachment point for the next amino acid of the peptide or such a linker is added to the element of the element-amino acid complex; contacting the two or more peptides with a binding element to form further element-peptide complexes with the next, now terminal amino acid of the peptide, tethering the further element-peptide complexes to the linker of the element-amino acid complex of (c) from the same peptide; and cleaving the element-peptide complexes from the peptides thereby providing linked element-amino acid complexes bound to the substrate; wherein the distance between the amino acids has been increased. In some embodiments of a method of any aforementioned aspect of the invention, the binding element includes a linker that provides an attachment point for the next amino acid of the peptide. In some embodiments of a method of any aforementioned aspect of the invention, the next amino acid is the terminal amino acid of the peptide after the peptide has been cleaved from the element-peptide complex. In certain embodiments of a method of any aforementioned aspect of the invention, the binding element comprises a linker. In some embodiments of a method of any aforementioned aspect of the invention also includes attaching to the binding element linker the next amino acid of the peptide after the peptide is cleaved from the element-peptide complex, resulting in the next amino acid of the peptide being part of an element-amino acid complex. In some embodiments of a method of any aforementioned aspect of the invention, the next amino acid of the peptide is a terminal amino acid of the peptide following the cleaving of the peptide from the element-peptide complex. In some embodiments of a method of any aforementioned aspect of the invention, the next amino acid of the peptide is part of element-amino acid complex. In certain embodiments of a method of any aforementioned aspect of the invention also includes attaching to the linker the next amino acid of the peptide that has been cleaved from the element-peptide complex, resulting in the next amino acid of the peptide being part of element-amino acid complex. In some embodiments of a method of any aforementioned aspect of the invention, the binding element binds to an N-terminal amino acid or N-terminal amino acid derivative of the peptide to form an element-peptide complex. In certain embodiments of a method of any aforementioned aspect of the invention, the binding element binds to a C-terminal amino acid or C-terminal amino acid derivative of the peptide to form an element-peptide complex. In certain embodiments of a method of any aforementioned aspect of the invention, prior to the step of tethering of the element-peptide complex to the substrate and/or the step of cleaving the element-peptide complex from the peptide, excess and/or unbound binding element is washed away. In some embodiments of a method of any aforementioned aspect of the invention, the steps of contacting the peptide with a binding element to form a further element-peptide complex with the next, now terminal amino acid of the peptide; tethering the further element-peptide complex to the linker of the element-amino acid complex; and cleaving the element-peptide complex from the peptide are repeated one or more times. In some embodiments of a method of any aforementioned aspect of the invention, the steps of contacting the peptide with a binding element to form a further element-peptide complex with the next, now terminal amino acid of the peptide; tethering the further element-peptide complex to the linker of the element-amino acid complex; and cleaving the element-peptide complex from the peptide are repeated for all amino acids of the peptide. In certain embodiments of a method of any aforementioned aspect of the invention, prior to contacting the peptide with the initial binding element, the peptide is affixed to a substrate. In certain embodiments of a method of any aforementioned aspect of the invention, prior to contacting the two or more peptides with the initial binding element, the two or more peptide are independently affixed to a substrate. In some embodiments, the two or more peptides are the same as each other. In some embodiments, at least two of the two or more peptides are different from each other. In certain embodiments, all of the two or more peptides are different from each other. In embodiments, the peptide and/or the two or more peptides are affixed to the substrate through the C′-terminal carboxyl group or a side chain functional group of the peptide. In some embodiments, the peptide and/or the two or more peptides are affixed to the substrate through the N′-terminal carboxyl group or a side chain functional group of the peptide. In some embodiments of a method of any aforementioned aspect of the invention, the peptide is covalently affixed to the substrate. In some embodiments of a method of any aforementioned aspect of the invention, the substrate is optically transparent. In some embodiments of a method of any aforementioned aspect of the invention, the substrate comprises a functionalized surface. In some embodiments of a method of any aforementioned aspect of the invention, the functionalized surface is selected from the group consisting of an azide functionalized surface, a thiol functionalized surface, alkyne, DBCO, maleimide, succinimide, tetrazine, TCO, vinyl, methylcyclopropene, a primary amine surface, a carboxylic surface, a DBCO surface, an alkyne surface, and an aldehyde surface. In some embodiments of a method of any aforementioned aspect of the invention, the method also includes sequencing the linearly expanded peptide. In some embodiments of a method of any aforementioned aspect of the invention, the method also includes the sequence of the peptide to a reference-protein-sequence-database. In some embodiments of a method of any aforementioned aspect of the invention, the method also includes comparing the sequences of each peptide, grouping similar peptide sequences and counting the number of instances of each similar peptide sequence. In some embodiments of a method of any aforementioned aspect of the invention, the peptide or the two or more peptides are from a sample. In some embodiments of a method of any aforementioned aspect of the invention, the sample includes a biological fluid, cell extract, tissue extract, or a mixture of synthetically synthesized peptides. In some embodiments of a method of any aforementioned aspect of the invention, the sample is a mammalian sample. In some embodiments of a method of any aforementioned aspect of the invention, the sample is a human sample. In some embodiments of a method of any aforementioned aspect of the invention, the binding element is a ClickT compound.

According to another aspect of the invention, an element-amino acid complex is provided, and includes: a binding element bound to one of 20 natural proteinogenic amino acids; a binding element bound to a post-translationally modified amino acid; or a binding element bound to a derivative of the one of 20 natural proteinogenic amino acids or a binding element bound to a derivative of the post-translationally modified amino acid.

According to another aspect of the invention, an element-amino acid complex binder is provided and includes a binder that binds to a subgroup of the 20 natural proteinogenic amino acids complexed with the binding element; a binder that binds to a subgroup of post-translationally modified amino acids complexed with the binding element; or a binder that binds to a derivative of the subgroup of the 20 natural proteinogenic amino acids or to a derivative of the subgroup of post-translationally modified amino acids. In some embodiments, the element-amino acid complex binder also includes a detectable label.

According to another aspect of the invention, an element-amino acid complex binder is provided, and includes a binder that binds to one of 20 natural proteinogenic amino acids complexed with the binding element; a binder that binds to a post-translationally modified amino acids complexed with the binding element; or a binder that binds to a derivative the one of the 20 natural proteinogenic amino acids or a binder that binds to a derivative of the post-translationally modified amino acid. In some embodiments, the element-amino acid complex binder also includes a detectable label.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 depicts a workflow for linearly expanding the distance between amino acids of a peptide using ClickT. The method described herein allows for linearly expanding the distance between some or all of the amino acids of a peptide while maintaining the sequence of the peptide.

FIG. 2A and FIG. 2B. FIG. 2A illustrates intramolecular expansion. FIG. 2B depicts how intramolecular expansion optimizes the environment around individual amino acids for amplification and detection.

FIG. 3A and FIG. 3B. FIG. 3A depicts the bonding of two amino acids in a peptide. As used herein, a “peptide” is defined as a protein and/or a string of two or more amino acids with a peptide bond. The chemical distance between amino acids is defined as the amount of chemical bonds between the amino group of one amino acid and the carboxyl group of the adjacent amino acid. In natural proteins and peptides, this distance is 1, as there is a single chemical bond that links the amino group and the carboxyl group between each amino acid.

FIG. 3B depicts the how the instantly claimed method increases the chemical bond distance to greater than 1 while still maintaining the order of amino acids of part or of the whole peptide. X = any element chemically conjugated between the group of one amino acid and the amine group of another amino acid.

### DETAILED DESCRIPTION

The present description provides compounds, compositions, methods, assays and reagents useful for linearly expanding a peptide. Peptides that have been expanded by the methods described herein are referred to as “linearly expanded peptides” or simply “expanded peptides”. As used herein, linearly expanding a peptide refers to increasing (expanding) the distance between amino acids of a peptide. The linear expanded peptide has the same amino acid sequence as the pre-expanded peptide except that the distance between the amino acids has been increased. As used herein, a “peptide” is defined as a protein and/or a string of two or more amino acids linked together by a peptide bond.

In one aspect, the methods are useful for linearly expanding a single peptide or multiple molecules of a single peptide. In one aspect, the methods are useful for linearly expanding multiple, distinct peptides.

In one aspect, the methods are useful for the simultaneous linear expansion of a plurality of single peptides.

Such linear expanded peptide or peptides can be useful as the basis of massively parallel sequencing techniques. As used herein, “sequencing” peptides in a broad sense involves observing the plausible identity and order of amino acids. In embodiments, sequencing involves observing the exact identity and order of amino acids of a peptide.

Additionally, the methods are useful for the simultaneous linear expansion of a plurality of distinct peptides. For example, samples comprising a mixture of different peptides, including proteins, can be expanded according to the methods described herein.

In embodiments, the expanded peptides can then be used, for example, to generate sequence information regarding individual peptides in the sample.

In embodiments, the expanded peptides can then be used, for example, for protein expression profiling in complex samples. For example, the expanded peptides can be useful for generating both quantitative (frequency) and qualitative (sequence) data for peptides, including proteins, contained in a sample.

In one embodiment, the invention allows for sequencing of proteins. The methods and reagents described herein can be useful for high-resolution interrogation of the proteome and enabling ultrasensitive diagnostics critical for early detection of diseases.

As used herein, the term “binding element” (also referred to herein as “element”) refers to any reagent that comprises a terminal amino acid reactive and, optionally, cleaving group; a tetherable group, and a connection point that allows for the attachment of a further element.

In embodiments, the binding element comprises a reactive group that binds to the terminal amino acid of the peptide; a tethering group that immobilizes the element-peptide complex to a physical substrate; a cleaving group that removes the element and bounded terminal amino acid from the peptide resulting in an element-amino acid complex; and a connection point for a linker group that allows for the attachment of further element bound amino acids (i.e., further element-amino acid complexes). In some embodiments, the element comprises the linker group. In some embodiments, the linker is added to the connection point after the element is bound to the terminal amino acid. In some embodiments, the linker is added to the connection point of the element of the element-amino acid complex.

The terminal amino acid reactive group reacts to and binds the terminal amino acid, or terminal amino acid derivative, of a peptide. When used for N-terminal amino acid linear expansion the terminal amino acid reactive group of the binding element comprises a primary amine reactive group that conjugates to the free amine at the N-terminal end of the peptide to form an element-peptide complex. When used for C-terminal amino acid linear expansion the terminal amino acid reactive group of the binding element comprises a C-terminal reactive group that conjugates to the modified or unmodified carboxylic group at the C-terminal end of the peptide to form an element-peptide complex.

In embodiments, the terminal amino acid reactive group is a primary amine reactive group. In one embodiment, the primary amine reactive group includes, but not limited to, isothiocyanate, phenyl isothiocyanate (PITC), isocyanates, acyl azides, N-hydroxysuccinimide esters (NHS esters), sulfonyl chlorides, aldehydes, glyoxals, epoxides, oxiranes, carbonates, aryl halides, imidoesters, carbodiimides, anhydrides, and fluorophenyl esters. In one embodiment, the reagent is phenyl isothiocyanate (PITC).

In embodiments, the N-terminal amino acid, or derivative thereof, and the binding element can be contacted under conditions that allow the N-terminal amino acid to conjugate to the primary amine reactive group of the binding element to form a complex.

In embodiments, the terminal amino acid reactive group is a C-terminal reactive group. In one embodiment, the C-terminal reactive group includes, but is not limited to, isothiocyanate, tetrabutylammonium isothiocyanate, diphenylphosphoryl isothiocyanate, acetyl chloride, cyanogen bromide, isothiocyanate, sodium thiocyanate, ammonium thiocyanate, and carboxypeptidases.

In embodiments, the C-terminal amino acid, or derivative thereof, and the binding element can be contacted under conditions that allow the C-terminal amino acid to conjugate to C-terminal reactive group of the binding element to form a complex.

In some embodiments, the binding element further comprises a cleaving group. In some embodiments, the cleaving group is the same as the terminal amino acid reactive group. In some embodiments, the functions of reacting to amines and cleaving the terminal amino acid from the peptide can be performed by the primary amine reactive group. In some embodiments, the primary amine reactive group having both of these functions includes, but is not limited to, isothiocyanate, phenyl isothiocyanate (PITC). In one embodiment, the primary amine reactive group is phenyl isothiocyanate (PITC). In one embodiment, the primary amine reactive group is isothiocyanate. In some embodiments, the functions of reacting to the C-terminus and cleaving amino acids can be performed by the same chemical group. In one embodiment, the C-terminal cleaving group is involved in the chemical removal of the terminal amino acid from the peptide to forms the ClickT-amino acid complex. In one embodiment, the cleaving group is isothiocyanate, tetrabutylammonium isothiocyanate, or diphenylphosphoryl isothiocyanate.

In embodiments, the terminal cleaving group is involved in the chemical removal of the terminal amino acid from the peptide. In one embodiment, the terminal cleaving group is involved in the chemical removal of the terminal amino acid from the peptide to form an element-amino acid complex. In embodiments, the cleaving group is PITC or isothiocyanate. In one embodiment, the cleaving group is assisted by engineered or wild type enzymes such as peptidases or proteases.

In embodiments, the element-amino acid complex is the binding element conjugated to the amino acid following cleavage from the peptide. In one embodiment, the element-amino acid complex can be chemically derivatized to be antigenic. In one embodiment the element-amino acid complex can be, but is not limited to, the following derivatized forms: thiazolone, thiohydantoin, or thiocarbamyl.

In embodiments, the tethering group includes, but is not limited to, isothiocyanate, tetrabutylammonium isothiocyanate, diphenylphosphoryl isothiocyanate, azide, alkyne, Dibenzocyclooctyne (DBCO), maleimide, succinimide, thiol-thiol disulfide bonds, Tetrazine, TCO, Vinyl, methylcyclopropene, a primary amine, a carboxylic acid an alkyne, acryloyl, allyl, and an aldehyde.

The tethering group can conjugate to a functionalized substrate such as a functionalized glass surface or integrated into a polymer network under conditions that allows for conjugation, thereby immobilizing the element-peptide complex on the substrate. Following cleave of the terminal amino acid from the peptide; the tethering group maintains the element-amino acid complex bound to the substrate.

In one embodiment, the binding element can tether directly to a functionalized surface of a substrate. For example, if the functionalize surface is an azide containing surface, then the binding element comprises a group that conjugates to azides, e.g., alkynes, and can tether directly to the surface. The conditional copper-catalyzed (Cu+) click chemistry of alkyne-azide bonds is bioorthgonal with a high yield and high reaction specificity suitable for isolating target molecule in complex biological environments.

The contacting and binding of components in a binding element complex, or a binding element complex-substrate complex can occur in a solvent including, but not limited to, aqueous solvents (such as water) or organic solvents (such as dioaxane, DMSO, THF, DMF, Toluene, acetonitrile).

In embodiments, the binding element conjugates to the terminal amino acid of the peptide to form the element-peptide complex. The element-peptide complex is then locally tethered to a physical substrate. The element-peptide complex is subsequently cleaved from the peptide resulting in an element-amino acid complex bound to the substrate. After cleavage, further element-amino acid complex(es) can optionally be linked to the element-amino acid complex bound to the substrate to allow for following consecutive rounds of linear expansion of the amino acids of the peptide.

In some embodiments, the binding element-amino acid complex is antigenic. In some embodiments, a portion of the binding element-amino acid complex is antigenic.

In embodiments of any of the compounds, compositions and/or methods described herein, the binding element has the structure of Formula I:

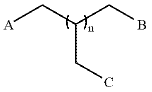

A is a terminal amino acid reactive and cleaving group;
B is a tetherable group;
C is a linker or attachment point for a linker; and
wherein n is is any number from 0 to 500. In one embodiment, n is any number from 0 to 250. In one embodiment, n is any number from 0 to 100. In one embodiment, n is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. In one embodiment, n is 0, 1, 2, 3, 4, 5, 6, 7, 8, 9, 10, 11, 12, 13, 14, 15, 16, 17, 18, 19, 20, 21, 22, 23, 24, or 25. In one embodiment, n is 0, 1, 2, 3, 4, 5, 6, 7, 8, 9, or 10. In one embodiment, n is 1, 2, 3, 4, or 5. In one embodiment, n is 1.

The compound of Formula I is also referred to herein as “ClickT”.

Formula II depicts a portion of one embodiment of a ClickT compound without the linker group. The linker group can be part of the ClickT compound or the linker can be added later to allow for the linking of additional ClickT-amino acid complex(es).

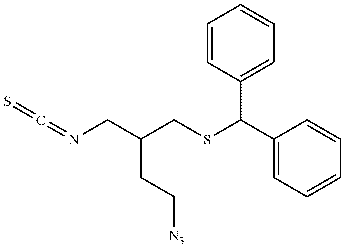 [SMILES: [N-]=[N+]=NCCC(CN=C=S)CSC(c1ccccc1)c1ccccc1]

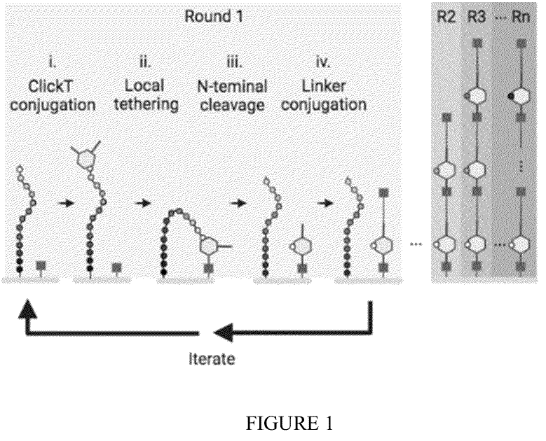

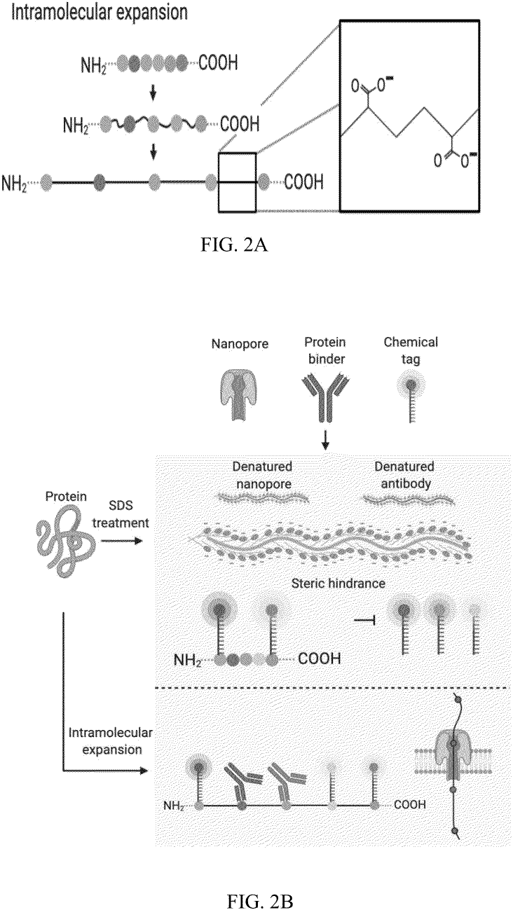

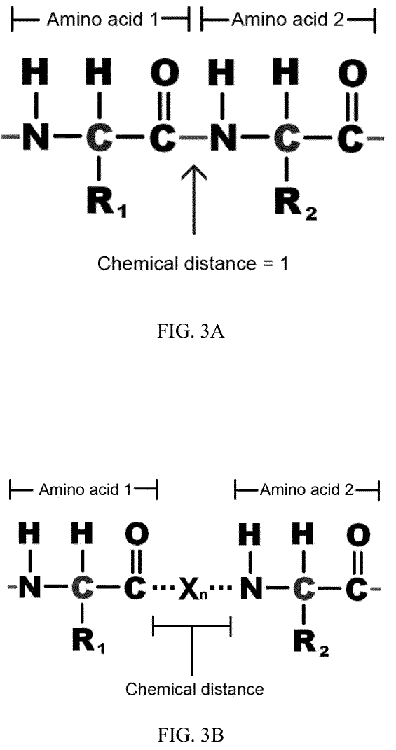

FIG. 1 shows a workflow of one example of a binding element binding to the terminal amino acid of a peptide to form an element-peptide complex. The tethering group conjugates to the element-peptide complex to a substrate. The element bound terminal amino acid is then cleave leaving an element-amino acid complex separately bound to the substrate. The cleaved element-terminal amino acid complex bound to the substrate can then be used as the starting point for binding further element bound amino acids of the peptide increase the distance between amino acids of the peptide.

In embodiments where the element comprises a linker that provides an attachment point for the next amino acid of the peptide or such a linker is added to the element of the element-amino acid complex, the peptide is again contacted with a binding element to form a further element-peptide complex with the next, now terminal amino acid of the peptide. The further element-peptide complex is then tethered to the linker of the previous element-amino acid complex bound to the substrate and then cleaved from the peptide thereby providing linked element-amino acid complexes bound to the substrate; wherein the distance between the amino acids has been increased.

The isolation of the terminal amino acid from the peptide allows for more selective and/or higher affinity binding of amino acids that is not influenced by the rest of the peptide. The linker, as either part of the element prior to contacting the peptide or added to the cleaved element-terminal amino acid complex, allows additional iterative rounds of linearization. This allows for sequential tethering of one element-amino acid complex to the next while maintaining the order of the amino acids indefinitely in a linear chain and providing spacing between the amino acids for independent detection and identification.

The present method internally disrupts the intramolecular properties of proteins by increasing the intramolecular distancing of its amino acids with charged molecules to enable single molecule protein sequencing to become successful. This strategy, intramolecular expansion, moves amino acids away from one another with charged linkers or analogous intermediates. More concretely, the present invention internally attaches charged linkers to amino acids one at a time, before detection (temporal separation), or between all amino acids in a chain (spatial separation), to overpower and disrupt the intrinsic intramolecular interactions between amino acids. Here, the charge disrupts the major hydrophobic and electrostatic interactions creating a protein’s structure, providing even accessibility across the whole protein. In addition, the additional amino-acid-to-amino-acid spacing, provided by the separation, will increase intramolecular spacing and reducing steric blockade between binders.

The invention provides a method for linearly expanding a peptide. As used herein, linear expanding a peptide means that the distance between amino acids of a peptide is increased (expanded) while maintaining the sequence of the peptide. In embodiments, the method comprises contacting the peptide with a binding element (also referred to herein as “the element”) that interacts with a terminal amino acid or a terminal amino acid derivative of the peptide to form an element-peptide complex, tethering the element-peptide complex to a substrate; cleaving the element-peptide complex from the peptide thereby providing an element-amino acid complex bound to the substrate. In embodiments, the element comprises a linker wherein the linker provides an attachment point for the next amino acid of the peptide. In embodiments, the method comprises attaching a linker to the element of the element-amino acid complex wherein the linker provides an attachment point for the next amino acid of the peptide. The “next amino acid of the peptide” is now the terminal amino acid and can be contacted with an element to form element-amino acid complex. Two or more element-amino acid complexes can be connected through the linker. In one embodiment, the peptide is affixed to a substrate.

In embodiments, the method is repeated one or more times. For example, after the terminal amino acid of the peptide has been removed, the peptide is again contacted with the element to form a further element-peptide complex with the next, now terminal amino acid, of the peptide; tethering the further element-peptide complex to the linker of the previous element; and cleaving the further element-peptide complex from the peptide. In embodiments, the element comprises a linker wherein the linker provides an attachment point for the next amino acid of the peptide. In embodiments, a further linker is attached to the further element-amino acid complex. The linker provides an attachment point for the use of the method on the next amino acid of the peptide. The “next amino acid of the peptide” is now the terminal amino acid and can be contacted with an element to form element-amino acid complex. Two or more element-amino acid complexes can be connected through the linker. In embodiments, the method is repeated until a portion of the peptide is expanded. In embodiments, the method is repeated until the entire peptide is expanded.

The invention also provides a method for linearly expanding two or more peptides. For example, the distance between amino acids of two or more peptides in a sample can be expanded (increased) while maintaining the sequences (i.e., order of amino acids) of the two or more peptides. In embodiments, the method comprises independently affixing the two or more peptides to a substrate; contacting the peptides with a binding element that interacts with the terminal amino acid or terminal amino acid derivative of each peptide to form an element-peptide complexes, tethering the element-peptide complexes to the substrate; cleaving the element-peptide complexes from the peptides thereby providing element-amino acid complexes bound to the substrate. In embodiments, the element comprises a linker wherein the linker provides an attachment point for the next amino acid of the peptide. In embodiments, the method comprises attaching a linker to the element of the element-amino acid complexes wherein the linker provides an attachment point for the next amino acid of the peptide. The “next amino acid of the peptide” is now the terminal amino acid and can be contacted with an element to form element-amino acid complex. Two or more element-amino acid complexes can be connected through the linker.

The invention also provides a method for linearly expanding of at least a portion of a peptide. In embodiments, the method comprises contacting the peptide with a binding element that interacts with a terminal amino acid or terminal amino acid derivative of the peptide to form an element-peptide complex, tethering the element-peptide complex to a substrate; cleaving the element-peptide complex from the peptide to form an element-amino acid complex bound to the substrate, wherein the element comprises a linker that provides an attachment point for the next amino acid of the peptide or such a linker is added to the element of the element-amino acid complex; again contacting the peptide with a binding element to form a further element-peptide complex with the next, now terminal amino acid of the peptide, tethering the further element-peptide complex to the linker of the previous element-amino acid complex; and cleaving the element-peptide complex from the peptide thereby providing linked element-amino acid complexes bound to the substrate; wherein the distance between the amino acids has been increased. In embodiments, the element of the further element-amino acid complex comprises a linker wherein the linker provides an attachment point for the next amino acid of the peptide. In embodiments, the method comprises attaching a linker to the element of the further element-amino acid complex wherein the linker provides an attachment point for the next amino acid of the peptide. The “next amino acid of the peptide” is now the terminal amino acid and can be contacted with an element to form element-amino acid complex. Two or more element-amino acid complexes can be connected through the linker. In embodiments, the method is repeated one or more times. In embodiments, the method comprises the linearly expanding all amino acids of the peptide.

The invention also provides a method for linearly expanding at least a portion of two or more peptides in a sample independently affixed attachment points on a substrate. In embodiments, the method comprises contacting the two or more peptides with a binding element that interacts with a terminal amino acid or terminal amino acid derivative of each peptide to form element-peptide complexes, tethering the element-peptide complexes to the substrate; cleaving the element-peptide complexes from the peptides to form element-amino acid complexes bound to the substrate, wherein the element comprises a linker that provides an attachment point for the next amino acid of the peptide or such a linker is added to the element of the element-amino acid complex; again contacting the peptides with a binding element to form a further element-peptide complex with the next, now terminal amino acid of the peptide, tethering the further element-peptide complex to the linker of the previous element-amino acid complex bound to the substrate; and cleaving the element-peptide complexes from the peptides thereby providing linked element-amino acid complexes bound to the substrate; wherein the distance between the amino acids has been increased.

In embodiments, the elements of the further element-amino acid complexes comprise a linker wherein the linker provides an attachment point for the next amino acid of the peptides. In embodiments, the method comprises attaching a linker to the elements of the further element-amino acid complexes wherein the linker provides an attachment point for the next amino acid of the peptide. The “next amino acid of the peptide” is now the terminal amino acid and can be contacted with an element to form element-amino acid complex. Two or more element-amino acid complexes can be connected through the linker. In embodiments, the method is repeated one or more times. In embodiments, the method comprises the linearly expanding all amino acids of the peptide. In some embodiments of the invention, the binding element comprises a linker that provides an attachment point for a next amino acid of the peptide after it has been cleaved from the element-peptide complex. In some embodiments, the method also includes attaching a linker to the element of the element-amino acid complex(es) and the linker provides an attachment point for a next amino acid of the peptide after the peptide has been cleaved from the element-peptide complex. Thus, the amino acid referred to as the next amino acid is a terminal amino acid of the peptide after the peptide has been cleaved from the element-peptide complex. In some embodiments, a method of the invention also includes attaching the next amino acid of the peptide after the peptide has been cleaved from the element-peptide complex to the linker. As a result, the next amino acid of the peptide is part of element-amino acid complex.

In embodiments of any of the methods disclosed herein, the methods optionally comprise washing away excess and/or unbound binding element prior to the step of cleaving the element-peptide complex from the peptide. Once a portion of the peptide, or all amino acids of the peptide, has been expanded by any of the processes described herein, the expanded peptide can be sequenced by any suitable method known in the art. Detection methods for protein sequencing include, but are not limited to, nanopores, ionic current nanopores, tunneling current nanopores, atomic force microscopy, protein binder, aptamer binder, multimeric binder, DNA-paint, and chemical conjugations.

In one embodiment, detecting and/or identifying the amino acid of the element-amino acid complex comprises contacting the element-amino acid complex with an element-amino acid complex binder, wherein the element-amino acid complex binder binds to an element-amino acid complex or a subgroup of element-amino acid complexes; and detecting the element-amino acid complex binder bound to the element-amino acid complex. Detecting binding of the binder to the element-amino acid complex allows for the identification of the terminal amino acid of the peptide.

In one embodiment, detecting and/or identifying the amino acid of the element-amino acid complex comprises contacting the element-amino acid complex with a plurality of element-amino acid complex binders, wherein each element-amino acid complex binder preferentially binds to a specific element-amino acid complex or a subgroup of element-amino acid complexes; and detecting the element-amino acid complex binder bound to the element-amino acid complex. By detecting the element-amino acid complex binder bound to the element-amino acid complex allows for identifying the terminal amino acid or subgroup of amino acids of the peptide. In embodiments, each element-amino acid complex binder preferentially binds to a specific element-amino acid complex. In embodiments, each element-amino acid complex binder binds to a subgroup of element-amino acid complexes.

It has been determined that the binding element described herein and element-amino acid complex binders can be used to generate sequence information by identifying the terminal amino acids of a peptide. The inventors have also determined that by first affixing the peptide molecule to a substrate, it is possible to determine the sequence of that immobilized peptide by iteratively detecting the element-amino acid complex at that same location on the substrate.

In one embodiment, detecting and/or identifying the amino acid of the element-amino acid complex can comprise direct detection through wavelengths of light. In one embodiment, Raman spectrum from single element-amino acid complexes are detected to identify the complex. In one embodiment, surface enhanced Raman spectroscopy is used to detect and/or identify the element-amino acid complex. In one embodiment, the Raman spectrum for each element-amino acid complex is distinguishable from one another. In one embodiment, the Raman spectrum for each element-amino acid complex are partially distinguishable from one another. In some embodiments, gold or silver can be deposited onto the substrate as a form of surface enhancement for Raman spectroscopy. In one embodiment, surface enhancement for Raman spectroscopy are nanoparticles that interact with element-amino acid complexes. In one embodiment, the interaction of the nanoparticles to element-amino acid complexes are, but not limited to, covalent, hydrophilic or hydrophobic interaction.

In embodiments of any of the compounds, compositions and/or methods disclosed herein, the binding element is a ClickT compound.

As used herein, the terms “peptide”, “polypeptide” or “protein” are used interchangeably herein and refer to two or more amino acids linked together by a peptide bond. The terms “peptide”, “polypeptide” or “protein” includes peptides that are synthetic in origin or naturally occurring. As used herein “at least a portion of the peptide” refers to two or more amino acids of the peptide. In some embodiments, a portion of the peptide includes at least 2, 3, 4, 5, 6, 7, 8, 9, 10, 15, 20, 30 or 50 amino acids (including any integer between 2 and 50), either consecutive or with gaps, of the complete amino acid sequence of the peptide, or the full amino acid sequence of the peptide.

The phrase “N-terminal amino acid” refers to an amino acid that has a free amine group and is only linked to one other amino acid by a peptide bond in the peptide. The phrase “N-terminal amino acid derivative” refers to an N-terminal amino acid residue that has been chemically modified, for example by an Edman reagent or other chemical in vitro or inside a cell via a natural post-translational modification (e.g., phosphorylation) mechanism, or a synthetic amino acid.

The phrase “C-terminal amino acid” refers to an amino acid that has a free carboxylic group and is only linked to one other amino acid by a peptide bond in the peptide. The phrase “C-terminal amino acid derivative” refers to a C-terminal amino acid residue that has been chemically modified, for example by a chemical reagent in vitro or inside a cell via a natural post-translational modification (e.g., phosphorylation) mechanism, or a synthetic amino acid.

The phrase “subgroup of element-amino acid complexes” refers to a set of amino acids that are bound by the same element-amino acid complex binder. In the broadest sense, the identity of the amino acid or subgroup is encoded in the binder. If the binder is not specific to one amino it may, for example, bind to two or three amino acids with some statistical regularity. This type of information is still relevant for protein identification since narrowing down the possibility of an amino acid is still relevant for database searches. Amino acid identity and binding variation is based on features like polarity, structure, functional groups and charge that can influence the specificity of the binder. Overall, the groups are based on the binder specificity and what they represent. A binder could bind two or more amino acids equally or with a varying degree of confidence, still providing sequence information.

As used herein, the binding of a binder to the element-amino acid complex or subgroup of element-amino acid complexes, refers to any covalent or non-covalent interaction between the binder and the element-amino acid complex. In one embodiment, the binding is covalent. In one embodiment, the binding is non-covalent.

As used herein, “sequencing a peptide” refers to determining the amino acid sequence of a peptide. The term also refers to determining the sequence of a segment of a peptide or determining partial sequence information for a peptide. Partial sequencing of a peptide is still powerful and sufficient to discriminate protein identity when mapped back to available databases. For example, it is possible to uniquely identify 90% of the human proteome by sequencing six (6) consecutive terminal amino acids of a protein. In instances where an element-amino acid complex binder that binds to a subgroup of element-amino acid complexes, the binders may not provide exact identity of the terminal amino acid but instead the plausible subgroup identity. Plausible sequence identity information is still powerful and sufficient to discriminate protein identity when mapped back to available databases.

As used herein, “affixed” refer to a connection between a peptide and a substrate such that at least a portion of the peptide and the substrate are held in physical proximity. The terms “affixed” or “tethered” encompass both an indirect or direct connection and may be reversible or irreversible, for example, the connection is optionally a covalent bond or a non-covalent bond.

In one embodiment, the substrate is a flat planar surface. In another embodiment, the substrate is 3-dimensional and exhibits surface features. In one embodiment, the surface is a functionalized surface. In some embodiments, the substrate is a chemically derivatized glass slide or silica wafer. In one embodiment, the substrate can be the peptide itself.

As used herein “the cleaving the N-terminal amino acid or N-terminal amino acid derivative of the peptide” refers to a chemical and/or enzymatical reaction whereby the N-terminal amino acid or N-terminal amino acid derivative is removed from the peptide while the remainder of the peptide remains affixed to the substrate.

As used herein “the cleaving the C-terminal amino acid or C-terminal amino acid derivative of the peptide” refers to a chemical and/or enzymatical reaction whereby the C-terminal amino acid or C-terminal amino acid derivative is removed from the peptide while the remainder of the peptide remains affixed to the substrate.

As used herein, the term “sample” includes any material that contains one or more polypeptides. Samples may be biological samples, such as biopsies, blood, plasma, organs, organelles, cell extracts, secretions, urine or mucous, tissue extracts and other biological samples of fluids either natural or synthetic in origin. The term sample also includes single cells. The sample may be derived from a cell, tissue, organism or individual that has been exposed to an analyte (such as a drug), or subject to an environmental condition, genetic perturbation, or combination thereof. The organisms or individuals may include, but are not limited to, mammals such as humans or small animals (rats and mice for example). In some embodiments, the sample is a biological sample from a plant.

In one embodiment, the attachment points on the functionalized surface are spatially resolved. As used herein, the term “spatially resolved” refers to an arrangement of two or more polypeptides on a substrate wherein chemical or physical events occurring at one polypeptide can be distinguished from those occurring at the second polypeptide. For example, two polypeptides affixed on a substrate are spatially resolved if a signal from a detectable label bound to one of the polypeptides can be unambiguously assigned to one of the polypeptides at a specific location on the substrate.

In one embodiment, peptides to be sequenced are affixed to a substrate. In some embodiments, the substrate is made of a material such as glass, quartz, silica, plastics, metals, hydrogels, composites, or combinations thereof. In one embodiment, the substrate is a flat planar surface. In another embodiment, the substrate is 3-dimensional. In some embodiments, the substrate is a chemically derivatized glass slide or silica wafer.

In one embodiment, the substrate is made from material that does not substantially affect the sequencing reagents and assays described herein. In one embodiment, the substrate is resistant to the basic and acidic pH, chemicals and buffers used for Edman degradation. The substrate may also be covered with a coating. In some embodiments, the coating is resistant to the chemical reactions and conditions used in Edman degradation. In some embodiments, the coating provides attachment points for affixing polypeptides to the substrate, and/or repelling non-specific probe adsorption. In some embodiments, the coating provides attachment points for tethering the element-peptide complex.

In some embodiments, the surface of the substrate is resistant to the non-specific adhering of polypeptides or debris, to minimize background signals when detecting the probes.

In one embodiment, the substrate is made of a material that is optically transparent. As used herein, “optically transparent” refers to a material that allows light to pass through the material. In one embodiment, the substrate is minimally- or non-autofluorescent.

In one embodiment, the peptides are affixed to the substrate. In one embodiment, the peptides are affixed to the substrate such that the N-terminal or C-terminal end of the peptide is free to allow the binding of the binding element. Accordingly, in some embodiments the peptide is affixed to the substrate through the N-terminal or C-terminal end of the peptide, the N-terminal amine or the C-terminal carboxylic acid group of the peptide. In some embodiments, the substrate contains one or more attachment points that permit a peptide to be affixed to the substrate.

In one embodiment, the peptides are affixed to the substrate such that the C-terminal end of the peptide is free to allow the binding of the binding element. Accordingly, in some embodiments the peptide is affixed to the substrate through the N-terminal end of the peptide, the N-terminal amine group or a side-chain-function group of the peptide. In some embodiments, the substrate contains one or more attachment points that permit a polypeptide to be affixed to the substrate.

In some embodiments, the peptide is affixed through a covalent bond to the surface. For example, the surface of the substrate may contain a polyethylene glycol (PEG) or carbohydrate-based coating and the peptides are affixed to the surface via an N-hydroxysuccinimide (NHS) ester PEG linker.

A number of different chemistries for attaching linkers and peptides to a substrate are known in the art, for example though not intended to be limiting, by the use of specialized coatings that include aldehydesilane, epoxysilane or other controlled reactive moieties. In one embodiment, the substrate is glass coated with Silane or related reagent and the polypeptide is affixed to the substrate through a Schiff’s base linkage through an exposed lysine residue.

In some embodiments, the peptide is affixed non-covalently to the substrate. For example, in one embodiment, the C-terminal end of the peptide is conjugated with biotin and the substrate comprises avidin or related molecules. In another embodiment, the C-terminal end of a peptide is conjugated to an antigen that binds to an antibody on the surface of the substrate. In another example, the N-terminal end of the peptide is conjugated with biotin and the substrate comprises avidin or related molecules. In another embodiment, the N-terminal end of a peptide is conjugated to an antigen that binds to an antibody on the surface of the substrate.

Additional coupling agents suitable for affixing a polypeptide to a substrate have been described in the art (See for example, Athena L. Guo and X. Y. Zhu. The Critical Role of Surface Chemistry In Protein Microarrays in Functional Protein Microarrays in Drug Discovery).

In one embodiment, there are provided element-amino acid complex binders that preferentially bind to a specific element-amino acid complex or a subgroup of element-amino acid complexes. As used herein, the phrase “preferentially binds to a specific ClickT-amino acid complex or a subgroup of element-amino acid complexes” refers to a binder with a greater affinity for a specific or subgroup of element-amino acid complexes compared to other specific or subgroup element-amino acid complexes. An element-amino acid complex binder preferentially binds a target element-amino acid complex or a subgroup of element-amino acid complexes if there is a detectable relative increase in the binding of the binder to a specific or subgroup of element-amino acid complexes.

In one embodiment, binders that preferentially bind to a specific element-amino acid complex or a subgroup of element-amino acid complexes are used to identify the N-terminal amino acid of a peptide. In one embodiment, binders that preferentially bind to a specific element-amino acid complex or a subgroup of element-amino acid complexes are used to sequence a peptide. In some embodiments, the binders are detectable with single molecule sensitivity.

In one embodiment, binders that preferentially bind to a specific element-amino acid complex or a subgroup of element-amino acid complexes are used to identify the C-terminal amino acid of a peptide. In one embodiment, binders that preferentially bind to a specific element-amino acid complex or a subgroup of element-amino acid complexes are used to sequence a peptide. In some embodiments, the binders are detectable with single molecule sensitivity.

In one embodiment, there are provided binders that selectively bind to an element-amino acid complex or an element-amino acid derivative complex. As used herein the phrase “selectively binds to a specific element-amino acid complex” refers to a binder with a greater affinity for a specific element-amino acid complex compared to other element-amino acid complexes. An element-amino acid complex binder selectively binds a target element-amino acid complex if there is a detectable relative increase in the binding of the binder to a specific element-amino acid complex.

In one embodiment, binders that selectively bind to an element-amino acid complex or an element-amino acid derivative complex are used to identify the N-terminal amino acid of a peptide and/or any amino acid in an expanded peptide of the invention. In one embodiment, binders that selectively bind to an element-amino acid complex or an element-amino acid derivative complex are used to sequence a polypeptide. In some embodiments, the binders are detectable with single molecule sensitivity.

In one embodiment, binders that selectively bind to an element-amino acid complex or an element-amino acid derivative complex are used to identify the C-terminal amino acid of a peptide and/or any amino acid in an expanded peptide of the invention. In one embodiment, binders that selectively bind to an element-amino acid complex or an element-amino acid derivative complex are used to sequence a peptide. In some embodiments, the binders are detectable with single molecule sensitivity.

The element-amino acid binders that target and recognize a specific element-amino acid complex or subgroup of element-amino acid complexes can be a protein or peptide, a nucleic acid a chemical or combination. The binders may also include components containing non-canonical amino acid and synthetic nucleotides. In one embodiment, a protein binder can be, but not limited to, an antibody, or an enzyme such as peptidases, proteases, aminoacyl tRNA synthetase, peptides or transport proteins like lipocalin. In one embodiment, the antibody is a polyclonal antibody. In one embodiment, the antibody is a monoclonal antibody. In one embodiment, a nucleic acid binder can be, but not limited to, an aptamer DNA, RNA or a mix of synthetic nucleotides. Aptamers are DNA/RNA with binding properties. In one embodiment, a chemical binder can be, but not limited to amino acid reactive chemistries such as maleimide and NHS ester, heterofunctional chemicals with 2 or more different functional groups, or non-covalently binding supramolecular chemistries.

In one embodiment, the plurality of binders may include 20 binders that each selectively bind to one of the 20 natural proteinogenic amino acids. In another embodiment, the binders include 20 binders that each selectively bind to a derivative of one of the 20 natural proteinogenic amino acids complexed with the binding element. In one embodiment, the derivatives are phenylthiocarbamyl derivatives. In a further embodiment, the binders include binders that selectively bind to post-translationally-modified amino acids or their derivatives complexed with the binding element. In one embodiment, the binders include binders that selectively bind to synthetic amino acids or their derivatives complexed with the binding element.

Detecting the binders bound to the element-amino acid complex can be accomplished by any detection method know by one of skill in the art.

In one embodiment, the binders include detectable labels. Detectable labels suitable for use with the present invention include, but are not limited to, labels that can be detected as a single molecule.

In one embodiment, the binders are detected by contacting the binders with a binder-specific antibody and the binder-specific antibody is then detected.

In some embodiments, the binders or labels are detected using magnetic or electrical impulses or signals.

In some embodiments, the labels on binders are oligonucleotides. Oligonucleotide labels are read out via any method known by one of skill in the art.

In one embodiment, the binders are detected by biological or synthetic nanopores via electrical impulses or signals.

In one embodiment, the labels are optically detectable, such as labels comprising a fluorescent moiety. Examples of optically detectable labels include, but are not limited to fluorescent dyes including polystyrene shells encompassing core dyes such as FluoSpheres™, Nile Red, fluorescein, rhodamine, derivatized rhodamine dyes, such as TAMRA, phosphor, polymethadine dye, fluorescent phosphoramidite, TEXAS RED, green fluorescent protein, acridine, cyanine, cyanine 5 dye, cyanine 3 dye, 5-(2'-aminoethyl)-aminonaphthalene-1-sulfonic acid (EDANS), BODIPY, 120 ALEXA or a derivative or modification of any of the foregoing. Additional detectable labels include color-coded nanoparticles, or quantum dots or FluoSpheres™. In one embodiment, the detectable label is resistant to photobleaching while producing lots of signal (such as photons) at a unique and easily detectable wavelength, with high signal-to-noise ratio.

One or more detectable labels can be conjugated to the binder reagents described herein using techniques known to a person of skill in the art. In one embodiment, a specific detectable label (or combination of labels) is conjugated to a corresponding binding reagent thereby allowing the identification of the binding reagent by means of detecting the label(s). For example, one or more detectable labels can be conjugated to the binding reagents described herein either directly or indirectly.

Binders bound to an element-amino acid complex affixed to the substrate are detected, thereby identifying the terminal amino acid of the polypeptide or protein. In one embodiment, the binder is identified by detecting a detectable label (or combination of labels) conjugated to the binder. Methods suitable for detecting the binders described herein therefore depend on the nature of the detectable label(s) used in the method.

In one embodiment, the binders or labels are repeatedly detected at that location using a high-resolution rastering laser/scanner across a pre-determined grid, unique position or path on a substrate. These methods are useful for the accurate and repeated detection of signals at the same coordinates during each sequencing cycle of the methods described herein. In some embodiments, the polypeptides are randomly affixed to the substrate and the detection of probes proceeds by repeatedly scanning the substrate to identify the co-ordinates and identities of probes bound to polypeptides affixed to the substrate.

In one embodiment, detecting the binders includes ultrasensitive detection systems that are able to repeatedly detect signals from precisely the same co-ordinates on a substrate, thereby assigning the detected sequence information to a unique polypeptide molecule affixed at that coordinate.

In one embodiment, the binders are detected using an optical detection system. Optical detection systems include a charge-coupled device (CCD), near-field scanning microscopy, far-field confocal microscopy, wide-field epi-illumination, light scattering, dark field microscopy, photoconversion, single and/or multiphoton excitation, spectral wavelength discrimination, fluorophore identification, evanescent wave illumination, total internal reflection fluorescence (TIRF) microscopy, super-resolution fluorescence microscopy, and single-molecule localization microscopy. In general, methods involve detection of laser-activated fluorescence using a microscope equipped with a camera, sometimes referred to as high-efficiency photon detection system. Suitable photon detection systems include, but are not limited to, photodiodes and intensified CCD cameras.

In one embodiment, examples of techniques suitable for single molecule detection of fluorescent probes include confocal laser (scanning) microscopy, wide-field microscopy, near-field microscopy, fluorescence lifetime imaging microscopy, fluorescence correlation spectroscopy, fluorescence intensity distribution analysis, measuring brightness changes induced by quenching/dequenching of fluorescence, or fluorescence energy transfer.

In one embodiment, the binding element complex is cleaved from the peptide. In one embodiment, cleaving exposes the terminus of the next, adjacent amino acid on the peptide, whereby the adjacent amino acid is available for reaction with a binding element. Optionally, the peptide is sequentially cleaved until the last amino acid in the peptide.

In some embodiments, the C-terminal amino acid is covalently affixed to the substrate and is not cleaved from the substrate. In one embodiment, cleaving exposes the N-terminus of an adjacent amino acid on the peptide, whereby the adjacent amino acid is available for reaction with a binding element. Optionally, the peptide is sequentially cleaved until the last amino acid in the peptide (C-terminal amino acid).

In some embodiments, the N-terminal amino acid is covalently affixed to the substrate and is not cleaved from the substrate. In one embodiment, cleaving exposes the C-terminus of an adjacent amino acid on the peptide, whereby the adjacent amino acid is available for reaction with a binding element. Optionally, the peptide is sequentially cleaved until the last amino acid in the peptide (N-terminal amino acid).

In one embodiment, sequential terminal degradation is used to cleave the N-terminal amino acid of the peptide. In one embodiment, sequential terminal degradation is used to cleave the C-terminal amino acid of the peptide. Degradation generally comprises two steps, a coupling step and a cleaving step. These steps may be iteratively repeated, each time removing the exposed terminal amino acid residue of a peptide.

In one embodiment terminal degradation proceeds by way of contacting the peptide with a suitable reagent such as PITC or a PITC analogue at an elevated pH to form a N-terminal phenylthiocarbamyl derivative. Reducing the pH, such by the addition of trifluoroacetic acid results in the cleaving the N-terminal amino acid phenylthiocarbamyl derivative from the polypeptide to form a free anilinothiozolinone (ATZ) derivative. This ATZ derivative may be detected. In one embodiment, ATZ derivatives can be converted to phenylthiohydantoin (PTH) derivatives by exposure to acid. This PTH derivative may be detected. In one embodiment, ATZ derivatives and PTH derivatives can be converted to phenylthiocarbamyl (PTC) derivatives by exposure to a reducing agent. This PTC derivative may be detected. In one embodiment, the pH of the substrate’s environment in controlled in order to control the reactions governing the coupling and cleaving steps.

In embodiments, terminal degradation proceeds by way of contacting the peptide with a suitable reagent such as ammonium thiocyanate after activation with acetic anhydride to form a C-terminal peptidylthiohydantion derivative. Reducing the pH, with a Lewis Acid results in the cleaving the C-terminal amino acid peptidylthiohydantion derivative by resulting in an alkylated thiohydantoin (ATH) leaving group from the polypeptide to form a free thiohydantion derivative. This ATH derivative may be detected. In one embodiment, ATH derivatives can be converted to thiohydantoin derivatives by exposure to acid. This thiohydantoin derivative may be detected. In one embodiment, the pH of the substrate’s environment in controlled in order to control the reactions governing the coupling and cleaving steps.

In one embodiment, the steps of contacting the peptide with a ClickT compound, wherein the ClickT compound binds to an N-terminal amino acid or N-terminal amino acid derivative to form a ClickT-peptide complex, tethering the ClickT-peptide complex to a substrate; cleaving the ClickT-peptide complex from the peptide resulting in a ClickT-amino acid complex bound to the substrate; are repeated in order to linear expand the distance between the amino acids of the peptide. Optionally, the steps are repeated at least 2, 5, 10, 20, 30, 50, or greater than 50 times in order to linear expand part of the peptide or the complete peptide.

In one embodiment, the steps of contacting the peptide with a ClickT compound, wherein the ClickT compound binds to an C-terminal amino acid or C-terminal amino acid derivative to form a ClickT-peptide complex, tethering the ClickT-peptide complex to a substrate; cleaving the ClickT-peptide complex from the peptide resulting in a ClickT-amino acid complex bound to the substrate; are repeated in order to linear expand the distance between the amino acids of the peptide. Optionally, the steps are repeated at least 2, 5, 10, 20, 30, 50, or greater than 50 times in order to linear expand part of the peptide or the complete peptide.

In one embodiment, the method further includes washing or rinsing the substrate before or after any one of the steps of affixing the substrate, contacting the peptide with a binding element, tethering the element-peptide complex to a substrate; or cleaving the element-peptide complex from the peptide. Washing or rinsing the substrate removes waste products such as debris or previously unused reagents from the substrate that could interfere with the next step in the method.

The methods described herein allow for the sequencing of very large number of peptide molecules on a single substrate or on a series of substrates. Accordingly, one aspect of the invention provides for sequencing a plurality of affixed peptides initially present in a sample. In one embodiment, the sample comprises a cell extract or tissue extract. In some embodiments, the methods described herein may be used to analyze the peptides contained in a single cell. In a further embodiment, the sample may comprise a biological fluid such as blood, urine or mucous. Soil, water or other environmental samples bearing mixed organism communities are also suitable for analysis.

In one embodiment, the sample comprises a mixture of synthetically synthesized peptides.

In one embodiment of the description, the method includes comparing the sequence of each peptide to a reference-protein-sequence database. In some embodiments, small fragments comprising 10-20 or fewer sequenced amino acid residues may be useful for detecting the identity of a peptide in a sample.

In one embodiment, the method includes de novo sequencing of peptides in order to generate sequence information about the peptide. In another embodiment, the method includes determining a partial sequence or an amino acid pattern and then matching the partial sequence or amino acid patterns with reference sequences or patterns contained in a sequence database.

In one embodiment, the method includes using the sequence data generated by the method as a molecular fingerprint or in other bioinformatic procedures to identify characteristics of the sample, such as cell type, tissue type or organismal identity.

In addition, as each peptide affixed to the substrate is optionally monitored individually, the method is useful for the quantitative analysis of protein expression. For example, in some embodiments, the method comprises comparing the sequences of each peptide, grouping similar peptide sequences and counting the number of instances of each similar peptide sequence. The methods described herein are therefore useful for molecular counting or for quantifying the number of peptides in a sample or specific kinds of peptides in a sample.

In a further embodiment, cross-linked peptides are sequenced using the methods described herein. For example, a cross-linked protein may be affixed to a substrate and two or more N-terminal amino acids are then bound and sequenced. The overlapping signals that are detected correspond to binders each binding the two or more terminal amino acids at that location. In one embodiment, it is possible to deduce or deconvolute the two multiplexed/mixed sequences via a computational algorithm and DB search.

In a further embodiment, the methods described herein are useful for the analysis and sequencing of phosphopeptides. For example, polypeptides in a sample comprising phosphopeptides are affixed to a substrate via metal-chelate chemistry. The phosphopolypeptides are then sequenced according to the methods described herein, thereby providing sequence and quantitative information on the phosphoproteome.

Additional multiplexed single molecule read-out and fluorescent amplification schemes can involve conjugating the binders with DNA barcodes and amplification with hybridized chain reaction (HCR). HCR involves triggered self-assembly of DNA nanostructures containing fluorophores and provides multiplexed, isothermal, enzyme-free, molecular signal amplification with high signal-to-background. HCR and branched DNA amplification can allow a large number of fluorophores to be targeted with single-barcode precision.

### EXAMPLES

### Example 1: Reagent for Amino Acid Recognition (“Binder” of the ClickT-amino Acid Complex)

Single-molecule peptide or protein sequence inherently involves elucidating the amino acid composition and order. All amino acids are organic small molecule compounds that contain amine (—NH2) and carboxyl (—COOH) functional groups, differentiated by their respective side chain (R group). The ability to identify all 20 amino acid requires a set of reagents or methods capable of discriminating their molecular structure with high specificity.

ClickT-based amino acid isolation solves the “local environment” problem, which is define as the interference of a binder’s ability to bind to a specific terminal amino acid due to the variability of adjacent amino acids. By removing the local environment problem with ClickT, binders are intended to target ClickT-amino acid complexes instead of the terminal amino acid.

To obtain more selective binders, portions of the ClickT-amino acid complexes can be used as small molecules for the development of antibodies with high affinity and specificity.

In one method, the ClickT-amino acid complexes can be injected into rabbits to elicit an immune response against the compounds and, thereby, the production of antibodies to bind the ClickT-amino acid complexes.

Downstream, the monoclonal antibodies generated via rabbit hybridoma technology will be tested for affinity, specificity and cross-reactivity. The antibodies secreted by the different clones will be assayed for cross-reactivity using enzyme-linked immunosorbent assay (ELISA) 29 and affinity will be measured using the label-free method BioLayer Interferometry (BLI) 30 for measuring the kinetics of protein-ligand interactions.

If antibodies do not display robust affinity or specificity towards ClickT bound amino acids, directed evolution approaches can be used for improving antibody affinity and specificity. Antibody binders can be engineered to target each amino acid isolated with ClickT using yeast display, a protein engineering technique that uses the expression of recombinant proteins incorporated into the cell wall of yeast to screen and evolve high affinity ligands. Yeast display has been used to successfully engineer antibodies that target small molecules with high affinity. The clones generated from the rabbit hybridoma can be used to construct an antibody library in yeast. The library will already have a bias towards the ClickT target so directed evolution via mutagenesis can introduce novel antibody variants with improved characteristics. Yeast Display is also capable of negative selection, which helps remove antibodies that cross-react with other targets. Negative selection would involve incubating yeast expressing the antibody library with magnetic beads conjugated to non-target antigens and pulling them out of solution. For example, when targeting ClickT bound to one particular amino acid, the other 19 amino acids can be negatively selected against to improve the odds of a highly specific binder.

In parallel, other binders such as enzymes or nucleic acid aptamers can be explored in case hybridoma technology does not generate any antibodies that target ClickT-bound amino acids. There exist 20 aminoacyl-tRNA synthetase enzymes that recognize their respective amino acids. Aminoacyl-tRNA synthetases or any other amino acid binding protein in nature can be used as scaffold proteins on yeast display and undergo directed evolution to select for specificity and affinity towards respective ClickT-bound amino acids. DNA/RNA aptamers are single-stranded oligonucleotides capable of binding various molecules with high specificity and affinity. It is established that RNA is able to form specific binding sites for free amino acids and that RNA aptamers have been evolved to change its binding specificity through repeated rounds of in vitro selection-amplification techniques of random RNA pools.

Antibody binders can simply have conjugated fluorophores or secondary antibodies conjugated to fluorophores that bind to the primary antibody, amplifying fluorescent intensity.

After binders are generated for targeting ClickT-bound amino acids, the sequencing scheme and imaging platform will be implemented on peptides, proteins and cell lysates.

### Example 2: Imaging and Scaling to Proteome

Amino acids can be identified by integrating all components of ClickT isolation of N-terminal amino acids, labeling with ClickT-amino acid specific binders, imaging, and subsequent cycles of amino acid identification. Sufficient cycles of amino acid identification will provide protein-sequencing information.

Peptides will first be immobilized to a substrate. For example, in N-terminal sequencing, peptides will first be immobilized by the C-terminus with carboxy crosslinking chemistry. Next, ClickT binds to the N-terminal amino acid of the peptide and tethers to a functionalized substrate. Following N-terminal cleavage, the isolated ClickT-bound amino acid is labeled with binders and imaged.

While this invention has been particularly shown and described with references to preferred embodiments thereof, it will be understood by those skilled in the art that various changes in form and details may be made therein without departing from the scope of the invention encompassed by the appended claims.

1. A method for linearly expanding a peptide comprising:
(a) contacting the peptide with a binding element that interacts with a terminal amino acid or a terminal amino acid derivative of the peptide to form an element-peptide complex;
(b) tethering the element-peptide complex to a substrate; and
(c) cleaving the element-peptide complex from the peptide resulting in an element-amino acid complex bound to the substrate.

2. (canceled)

3. The method according to claim 1, wherein the binding element comprises a linker that provides an attachment point for anext amino acid of the peptide cleaved from the element-peptide complex.

4. The method according to claim 1, further comprising (d) attaching a linker to the element of the element-amino acid complexes wherein the linker provides an attachment point for a next amino acid of the peptide cleaved from the element-peptide complex, wherein the next amino acid is a terminal amino acid of the cleaved peptide.

5. The method according to claim 3, further comprising attaching to the linker the next amino acid of the peptide cleaved from the element-peptide complex, so the next amino acid of the peptide is part of element-amino acid complex.

6-8. (canceled)

9. The method according to claim 3, wherein steps (a) through (c) are repeated one or more times, and optionally steps (a) through (c) are repeated for all amino acids of the peptide.

10. (canceled)

11. The method according to claim 4, wherein steps (a) through (d) are repeated one or more times, and optionally steps (a) through (d) are repeated for all amino acids of the peptide.

12. (canceled)

13. The method according to claim 1, wherein, prior to (a), the peptide is affixed to a substrate.

14-20. (canceled)

21. The method according to claim 1, wherein the substrate comprises a functionalized surface.

22. (canceled)

23. A method for linearly expanding of at least a portion of a peptide comprising:
(a) contacting the peptide with a binding element that interacts with a terminal amino acid or terminal amino acid derivative of the peptide to form an element-peptide complex;
(b) tethering the element-peptide complex to a substrate;
(c) cleaving the element-peptide complex from the peptide to form an element-amino acid complex bound to the substrate, wherein the element comprises a linker that provides an attachment point for a next amino acid of the peptide or such a linker is added to the element of the element-amino acid complex;
(d) contacting the peptide with a binding element to form a further element-peptide complex with the next, now terminal amino acid of the peptide,
(e) tethering the further element-peptide complex to the linker of the element-amino acid complex of (c); and
(f) cleaving the element-peptide complex from the peptide thereby providing linked element-amino acid complexes bound to the substrate;
wherein the distance between the amino acids has been increased.

24. (canceled)

25. The method according to claim 23, wherein the binding element comprises a linker that provides an attachment point for the next amino acid of the peptide cleaved from the element-peptide complex.

26. The method according to claim 23, further comprising step (g) attaching a linker to the element of the element-amino acid complexes wherein the linker provides an attachment point for the next amino acid of the peptide cleaved from the element-peptide complex, and the next amino acid is a terminal amino acid of the cleaved peptide.

27. The method according to claim 23, further comprising attaching to the linker the next amino acid of the peptide, so the next amino acid of the peptide is part of element-amino acid complex.

28-30. (canceled)

31. The method according to claim 23, wherein steps (d) through (f) are repeated one or more times.

32. The method according to claim 31, wherein steps (d) through (f) are repeated for all amino acids of the peptide.

33. The method according to claim 23, wherein, prior to (a), the peptide is affixed to a substrate.

34-49. (canceled)

50. An element-amino acid complex binder comprising:
(a) a binder that binds to one or a subgroup of the 20 natural proteinogenic amino acids complexed with a binding element;
(b) a binder that binds to one or a subgroup of post-translationally modified amino acids complexed with the binding element; or
(c) a binder that binds to a derivative of (a) or (b).

51-52. (canceled)

53. The method of claim 1, further comprising performing steps (a) through (c) on one or more additional peptides thereby linearly expanding the two or more peptides.

54. The method according to claim 53, wherein, prior to step (a), the two or more peptides are independently affixed to the substrate.

55. The method according to claim 54, wherein the two or more peptides are different.

56. The method of claim 23, further comprising performing steps (a) through (f) on one or more additional peptides, thereby linearly expanding at least a portion of the two or more peptides.
